SEQUENCE LISTING (16 sequences)

SEQ ID NO: 1 - DNA, 34 bp, Artificial Sequence
  h10RKCS plus strand primer for the PCR amplification, from plasmid pSW8.1, of the human IL-10 receptor
ccataagcttgccaccatgctgccgtgcctcgta

SEQ ID NO: 2 - DNA, 50 bp, Artificial Sequence
  h10R6H minus strand primer for the PCR amplification, from plasmid pSW8.1, of the human IL-10 receptor
gcaggatccttagtgatggtgatggtgatggttggtcacggtgaaatact

SEQ ID NO: 3 - DNA, 708 bp, Homo sapiens
atgctgccgtgcctcgtagtgctgctggcggcgctcctcagcctccgtcttggctcagacgctcatgggacagagctgcccagccctccgtctgtgtggtttgaagcagaatttttccaccacatcctccactggacacccatcccaaatcagtctgaaagtacctgctatgaagtggcgctcctgaggtatggaatagagtcctggaactccatctccaactgtagccagaccctgtcctatgaccttaccgcagtgaccttggacctgtaccacagcaatggctaccgggccagagtgcgggctgtggacggcagccggcactccaactggaccgtcaccaacacccgcttctctgtggatgaagtgactctgacagttggcagtgtgaacctagagatccacaatggcttcatcctcgggaagattcagctacccaggcccaagatggcccccgcgaatgacacatatgaaagcatcttcagtcacttccgagagtatgagattgccattcgcaaggtgccgggaaacttcacgttcacacacaagaaagtaaaacatgaaaacttcagcctcctaacctctggagaagtgggagagttctgtgtccaggtgaaaccatctgtcgcttcccgaagtaacaaggggatgtggtctaaagaggagtgcatctccctcaccaggcagtatttcaccgtgaccaacgtc

SEQ ID NO: 4 - DNA, 34 bp, Artificial Sequence
  h10RIA3 minus strand primer for the PCR amplification, from plasmid pSW8.1, of the human IL-10 receptor
cgtacgtcgacgttggtcacggtgaaatactgcc

SEQ ID NO: 5 - DNA, 32 bp, Artificial Sequence
  higAM minus strand primer for the PCR amplification, from cell line DAKIKI cDNA, of the human IgA1 hinge, CH2, and CH3 domains
cgctggatcctcagtagcaggtgccgtccacc

SEQ ID NO: 6 - DNA, 30 bp, Artificial Sequence
  higAP2 plus strand primer for the PCR amplification, from cell line DAKIKI cDNA, of the human IgA1 hinge, CH2, and CH3 domains
ctacgcgtcgacgttccctcaactccacct

SEQ ID NO: 7 - DNA, 32 bp, Artificial Sequence
  higG1P plus strand primer for the PCR amplification, from cell line ARH-77 cDNA, of the human IgG1 hinge, CH2, and CH3 domains
ctacgcgtcgacaaaactcacacatgcccacc

SEQ ID NO: 8 - DNA, 32 bp, Artificial Sequence
  higGM minus strand primer for the PCR amplification, from cell line ARH-77 cDNA, of the human IgG1 hinge, CH2, and CH3 domains
cgctggatcctcatttacccggagacagggag

SEQ ID NO: 9 - DNA, 7 bp, Artificial Sequence
  Synthetic transcription termination signal
tttttat

SEQ ID NO: 10 - DNA, 24 bp, Artificial Sequence
  Rat K1 primer used for the reverse transcription of JES5 RNAcomplementary to the 3′ region of the rat kappa constant region
ctcattcctgttgaagctcttgac

SEQ ID NO: 11 - DNA, 21 bp, Artificial Sequence
  RatGl primer used for the reverse transcription of JES5 RNAcomplementary to the CH2 region of rat IgG1
ggagtcagagtgatggtgagc

SEQ ID NO: 12 - DNA, 35 bp, Artificial Sequence
  RatG2 primer, used as the minus strand primer for PCR-amplification of the polydG-tailed cDNA, complementary to the CH1/hinge boundary of rat IgG1
gcgggatcctaggcacaattttcttgtccaccttg

SEQ ID NO: 13 - DNA, 44 bp, Artificial Sequence
  RatK2 primer used as the minus strand primer for the PCR amplification of the polydG-tailed cDNA, complementary to the 3′ region of the kappa constant region
cgcggatcctaacactcattcctgttgaagctcttgacgacggg

SEQ ID NO: 14 - DNA, 35 bp, Artificial Sequence
  3GT used as plus strand primer in PCR amplification of the gamma and kappa chain polydG-tailed JES5 cDNAs
cctactcgagtcgacaagcttcccccccccccccc

SEQ ID NO: 15 - DNA, 40 bp, Artificial Sequence
  Rat G3 primer constructed following sequencing of thePCR-amplified polydG JES5 gamma
cgctaagcttgccaccatgaaatgcagctggatcatcctc

SEQ ID NO: 16 - DNA, 40 bp, Artificial Sequence
  RatK3 primer constructed following sequencing of thePCR-amplified polydG JES5 kappa
cgctaagcttgccaccatggacatgagggcccatgctcag

(19) United States
(12) Patent Application Publication
(10) Pub. No.: US 2007/0178065 A1
(43) Pub. Date: Aug. 2, 2007

(54) Neutralizing factors as vaccine adjuvants

(76) Inventors: Edmund C. Lattime, Princeton, NJ (US); Claude Monken, Lawrenceville, NJ (US)

Correspondence Address: Licata & Tyrrell P.C., 66 E. Main Street, Marlton, NJ, 08053, US

(21) Appl. No.: 11/492,293

(22) Filed: Jul. 25, 2006

Related U.S. Application Data
(63) Continuation-in-part of application No. 10/138,783, filed on May 3, 2002, now abandoned.

(51) Int. Cl. A61K 48/00; C12N 15/86

(52) U.S. Cl. 424/93.2; 435/456

(57) ABSTRACT

The present invention is a vaccine adjuvant composed of a vaccinia virus vector that encodes a polypeptides capable of neutralizing immune suppressive factors thereby enhancing or stimulating an immune response to a vaccine.

This application is a continuation-in-part application of U.S. patent application Ser. No. 10/138,783, filed May 3, 2002, the content of which is incorporated herein by reference in its entirety.

The invention was made with government support under grant R01-CA42908 awarded by the National Institutes of Health and grant RO1-CA55322 awarded by the National Cancer Institute. The United States government may have certain rights in this invention.

### BACKGROUND OF THE INVENTION

The induction of an immune response is a complex process requiring the recruitment of appropriate immune cells to the site of the foreign pathogen (such as a tumor cell) and, importantly, the interplay of a variety of immune modulatory molecule, such as cytokines, which not only control the induction and magnitude of the response but also its nature, i.e., the production of antibodies and the activation of cells that reject tissue and destroy infected and neoplastic cells. The primary goal of tumor immunotherapy is to modulate the immune system so as to alert immune effector cells to the presence of tumor tissue and to elicit immune reactions that selectively destroy the tumor cells.

Traditional immunotherapeutic strategies have included the immunization of subjects with killed tumor cells or tumor antigens to enhance host immune responses against the tumor, ex vivo transfection of tumor cells with pro-immune cytokines or costimulatory molecules followed by reinjection of the tumor cells into the ohst, system ic administration of cytokines, nonspecific stimulation of the i9mmune system by local administration of inflammatory substances such as bacillus Calmette-Guerin mycobacterium, adoptive cellular immunotherapy using a host's peripheral blood or tumor infiltrating lymphocytes expanded in culture and reinjected, as well as passive immunotherapy by administration of monoclonal antibodies (Abbas (2000) Cellular and Molecular Immunology, 4^{th} Ed., Saunders, Chapter 17).

From these and other studies of immune responses to tumors, it has become apparent that the immune system is capable of recognizing tumors and there is substantial evidence that the immune system responds to many tumors even in the absence of immunostimulatory therapies. Histopathologic studies have shown that many tumors and their metastases are surrounded by infiltrates consisting of T cells, natural killer cells, and macrophages (Soiffer, et al. (1998) Proc. Natl. Acad. Sci. USA 95:13141-13146). However, most of these infiltrates fail to induce more than modest inflammatory reactions within tumors and ordinarily do not result in the destruction or regression of the tumors or their metastases. These observations have led to the recognition that tumors may not need to evade recognition by the immune system entirely in order to proliferate, but instead may use specialized mechanisms to counter tumor-specific immune responses, thereby rendering them ineffectual.

Indeed, a number of recent investigations have demonstrated that some tumors go far beyond passive immune evasion and actively engage in immune suppression to promote their growth despite an alerted immune system. For example, many tumors secrete large quantities of TGF-β, a potent inhibitor of lymphocyte and macrophage proliferation (Robbins (1994) Pathologic Basis of Disease, 5th Ed., Saunders, Chapter 7), whereas other tumors have been demonstrated to express FasL, a cell surface molecule capable of triggering cell death in tumor-infiltrating T cells (Hahne, et al. (1996) Science 274:1363-66; Williams (1996) Science 274:1302). Similarly, certain prostaglandins are known to inhibit T-cell activation (Kolenko, et al. (1999) Blood 93(7):2308-2318). Interleukin-10 (IL-10), another factor recently discovered to be immune suppressive, is produced by a number of different tumors and has been shown to interfere with antigen-induced T cell proliferation (de Waal Malefyt, et al. (1991) J. Exp. Med. 174:915-924). It has further been shown that a tumor cell line that does not itself express IL-10 is nevertheless able to induce infiltrating or neighboring cells to produce IL-10, thereby preventing the generation of an immune response directed at a tumor-associated antigen (Halak, et al. (1999) Cancer Res. 59:911-917).

These and other mechanistic studies have demonstrated a need to overcome immune suppressive factors found in the tumor microenvironment and perhaps systemically in order to elicit an effective anti-tumor response. A variety of tumors are known to either express or to induce the expression of factors that suppress tumor-specific immune responses at the tumor site. In addition, patients in the advanced stages of cancer often exhibit a marked immunosuppression characterized by abnormalities in T cell receptor structure, T cell signaling and signal transduction pathways, the etiology of which may be the systemic secretion of soluble immune suppressive factors due to large tumor burden (Ostrand-Rosenberg, et al. (1999) Gene Therapy of Cancer, Academic Press, Chapter 3). Studies have identified a number of tumor-secreted or tumor-associated immune suppressive factors the inhibition of which may restore normal immune functions and render tumors susceptible to eradication by the host immune system. These tumor-associated factors may not only act at the tumor site to suppress antitumor immunity but may also act systemically to inhibit the ability of tumor antigen encoding vaccines to induce effective antitumor immunity. A strategy for neutralizing immune suppressive factors such as IL-10, IL-4, VEGF, TGF-β, prostaglandins, and other immune suppressive molecules identified at the tumor site, is therefore expected to overcome the tumor-associated immune suppression and to allow the development of a productive antitumor immune response.

Thus, it is apparent that there is a need to inhibit the activity of immune suppressive factors to allow the generation of an effective immune response by the immune system. Immunotherapy aimed solely at stimulating the immune system may increase recognition and detection of tumors or antigens by the immune system, but by itself does not address the counter-offensive mechanisms employed to suppress cell-mediated immune responses. An approach aimed at inactivating or neutralizing immune suppressive factors that blocks or adversely modulates the immune system's response would be very useful in the immunotherapy of cancer as well as pathogens.

### SUMMARY OF THE INVENTION

The present invention is a vaccine adjuvant composed of a vaccinia virus vector encoding at least one polypeptide that neutralizes an immune suppressive factor.

The present invention is also a method for enhancing an immune response to a vaccine by administering the vaccine in combination with the vaccine adjuvant of the invention to a subject so that the cells of the subject express the at least one polypeptide thereby enhancing an immune response to the vaccine in the subject. In certain embodiments, the vaccine and vaccine adjuvant are co-administered with a second vaccinia virus vector encoding an immune active cytokine.

A kit containing a formulated vaccine of the invention is also provided.

### BRIEF DESCRIPTION OF THE DRAWINGS

FIG. 1 (upper panel) shows that the immunoadhesin constructed from the human IL-10 receptor and an IgG backbone (human IL-10R adhesin) strongly binds human IL-10 as evidenced by the low recovery of IL-10 (second bar) compared to the almost complete recovery of human IL-10 when applied to the murine IL-10 and IL-4 receptor IgG immunoadhesins (bars 2 and 3).

FIG. 1 (lower panel) shows and that both the ligand binding domain of the receptor (human extracellular IL-10R) and the IL-10 receptor IgG immunoadhesin (human IL-10 R adhesin) sequester IL-10 and inhibit the proliferation of the hIL-10-responsive cell line Ba8.1 as evidenced by the decrease in O.D. values in the MTT assay. By contrast, neither media alone (control) nor murine IL-10 R immunoadhesin have any significant effect on the proliferation of the IL-10 responsive cells.

FIG. 2 demonstrates that the extracellular domain of the human IL-10 receptor (human IL-10R-6xHis) strongly and specifically binds and removes human IL-10 from media (middle bar), while the murine IL-10 receptor immunoadhesin is unable to remove human IL-10 from media (third bar).

FIG. 3 (upper panel) shows that the murine IL-4 receptor immunoadhesin construct binds murine IL-4 with specificity (middle bar) and removes IL-4 from media, while the murine IL-10 receptor immunoadhesin control does not significantly bind to murine IL-4 (third bar).

FIG. 3 (lower panel) shows that murine IL-4 receptor immunoadhesin specifically binds to murine IL-4 in a dose-dependent fashion (bars three and four) and thereby inhibits its activity in ELISA, while the human IL-10 receptor immunoadhesin does not bind to murine IL-4 (second bar).

FIG. 4 shows the dual vector containing the nucleotide sequences encoding the constant regions of the kappa and gamma chains of the rat anti-murine IL-10 monoclonal antibody, JES5.

FIG. 5 shows that the murine IL-10 antibody construct binds to murine IL-10 in a dose-dependent fashion and inhibits the activity of murine IL-10 in ELISA.

FIG. 6 shows that the human IL-10 receptor IgA immunoadhesin (human IL-10R adhesin) sequesters human IL-10 from media and inhibits the proliferation of the IL-10 responsive cell line Ba8.1 as measured by the MTT assay. Murine IL-10 receptor IgA immunoadhesin, by contrast, binds only slightly to the human IL-10 in the supernatant and thus does not significantly inhibit cell proliferation.

FIG. 7 shows that the human IL-10 receptor IgA immunoadhesin binds IL-10 and inhibits its activity in ELISA, whereas murine IL-10 receptor IgA immunoadhesin has virtually no effect on human IL-10 activity.

FIG. 8 shows the plasmid map for pSC65 (GenBank Accession #AX003206).

FIG. 9 shows the modifications made to pSC65 to produce the dual gene recombinant plasmid, pVTK2SEL, as well as insertion of the antibody heavy and light chains to generate the rat anti-mouse monoclonal IL-10 antibody recombination vector, pVJES5GK.

FIG. 10 shows the promoter and multiple cloning site map for the dual gene recombination vector, pVTK2SEL.

FIG. 11 shows a plasmid map of the dual gene recombination vector, pVTK2SEL.

FIG. 12 shows that supernatant from a cell infected with a vaccine virus encoding anti-IL10 antibody neutralizes mIL1.

FIG. 13 shows that vaccinia virus expressing anti-IL-10 antibody enhances antitumor activity of a vaccine composed of vaccinia virus expressing Uty tumor antigen.

### DETAILED DESCRIPTION OF THE INVENTION

It has now been found that neutralization of immune suppressive factors provides an adjuvant effect for stimulating an immune response to vaccine antigens. Specifically, it has been demonstrated that the administration of a vaccinia virus vector encoding an anti-IL-10 antibody enhances antitumor activity to a tumor antigen-encoding vaccine and a vector encoding an IL-10 immunoadhesin has the ability to inhibit IL-10-responsive cell proliferation. Therefore, vaccinia virus vectors encoding polypeptides such as antibodies or immunoadhesins which neutralize the activity of immune suppressive factors, such as IL-4, IL-10, VEGF, TGF-β, or prostaglandins, are useful as adjuvants for stimulating an immune response to vaccines.

Accordingly, the present invention is a gene delivery vector designed to encode a neutralizing factor for expression of the neutralizing factor by cells. This vector is preferably equipped with regulatory sequences operably linked to the sequences coding for the neutralizing factors to drive their expression from cells in sufficient amounts so as to lead to the inhibition of immune suppressive factors. The regulatory sequences that drive the expression of the sequences coding for a neutralizing factor can be modified to be inducible or tissue/cell-type specific. Inducible promoters allow the external control of the timing and duration of gene expression. Examples of inducible promoters suitable for use in gene delivery vectors are tetracycline-responsive promoters (Gossen and Bujard (1992) Proc. Natl. Acad. Sci. USA 89:5547-5551), or a synthetic progesterone antagonist-inducible promoter (Wang, et al. (1994) Proc. Natl. Acad. Sci. USA 91:8180-8184). Tissue-specific promoters can confine the expression of the delivered genes to tumor cells and normal cells of a specific lineage. Examples of tissue-specific promoters among many others known to persons skilled in the art are the insulin promoter specific to the beta islet cells of the pancreas, the whey acidic protein promoter specific to the breast, the tyrosinase promoter specific to melanocytes, the Ren-2 promoter specific to the kidney, the von Willebrand factor promoter specific to endothelial cells, and the albumin promoter specific to the liver.

Examples of suitable viral gene delivery vectors known in the art are retroviruses (including Moloney murine leukemia virus, lentiviruses and foami viruses), adenoviruses, parvoviruses (including adeno-associated virus), herpes simplex viruses, human cytomegalovirus, Epstein-Barr virus, poxviruses (vaccinia, MVA and fowlpox), negative-strand RNA viruses (including influenza) and alphaviruses, among others.

Nonviral vectors suitable for delivery of the neutralizing constructs include cationic liposomes (such as mixtures of DOPE with DOTMA, DOSPA, DDAB, DOGS, DOTAP, DMRIE and DC cholesterol), DNA-protein complexes, DNA complexed with biocompatible polymers such as polysaccharides or atecollagen, receptor-mediated polylysine-DNA complexes, and mechanical administration of naked DNA. The administration of the naked DNA can be performed in a number of different ways known to those of skill in the art, such as by lipofection, direct DNA injection, particle mediated transfer (gene gun), DNA ligand, or administration of DNA linked to killed adenovirus (Cooper, Chapter 5, Gene Therapy of Cancer, Academic Press, 1999). In addition, combinations of vector systems, such as hybrid adenoviral/retroviral vectors, hybrid alphavirus/retroviral vectors or combinations of viral vectors with nonviral gene delivery systems, such as adenovirus used in conjunction with LIPOFECTAMINE, poloxamer 407 or polyethylene glycol, as well as plasmid DNA vectors encoding viral replicons may be suitable delivery vehicles.

The gene delivery vectors may further be used in conjunction with pharmaceutically acceptable carriers. Examples of such pharmaceutically acceptable carriers are saline solutions, buffered solutions, emulsions, or suspensions among many others. Pharmaceutically acceptable carriers for therapeutic use are well known in the pharmaceutical art and are described, for example in Remington's Pharmaceutical Sciences ((1990) Gennaro Ed., 18th Edition, Mack Publishing Co., Easton, Pa.). Such carriers may be selected in accordance with the intended route of administration and the standard pharmaceutical practice.

In certain embodiments, the gene delivery vector is delivered via oral, rectal, topical, buccal (e.g., sub-lingual), vaginal, parenteral (e.g., subcutaneous, intradermal, intravenous, intraperitoneal), topical (i.e., both skin and mucosal surfaces, including airway surfaces), intranasal, transdermal, intraarticular, intrathecal and inhalation routes or can alternatively be directly injected into a palpable tumor mass, or in the case of internal tumors, be injected percutaneously into the tumor with the guidance of noninvasive imaging technology known in the art (such as Magnetic Resonance Imaging (MRI), Positron Emission Tomography (PET), other nuclear imaging techniques, X-rays, Computed Tomography, optical absorption or ultrasonography), or in the case of bladder cancer, administered intravesically. Similarly, the vectors can be injected into the tumors by the use of image-guided endoscopy, bronchoscopy or cystoscopy. When administered intravenously, the instant vectors can be modified to home to tumor tissue by conjugation with ligands that bind to specific cellular receptors or when tissue- or tumor-specific regulatory sequences are used for the expression of the genes encoding the neutralizing factors.

In one embodiment, the neutralizing factors are constructed from the extracellular (ligand binding) domains of the receptors for those immune suppressive factors that are present in a tumor microenvironment or systemically. The DNA sequence coding for the extracellular domain of the receptor for a particular immune suppressive factor is identified and engineered into a suitable vector for administration and delivery to cells, followed by the expression and secretion of the soluble receptor by the vector-transfected cells either systemically or in the local tumor environment, where the receptor binds and neutralizes its cognate immune suppressive factor by preventing the interaction between the immune suppressive factor and its native receptor on immune effector cells.

Alternatively, any protein domain containing a binding domain (such as non-receptor binding domains, antigen binding sites of antibodies, or binding sites of enzymes) specific for an immune suppressive factor may be identified using standard techniques known to those skilled in the art, such as by sequence homology comparison (Johnson and Church (2000) Proc. Natl. Acad. Sci. USA 97(8) :3965-3970), predictive techniques based on known protein structure (Lichtarge, et al. (1996) J. Mol. Biol. 257(2):342-358; Peters, et al. (1996) J. Mol. Biol. 256:201-213) and in vitro protein-protein interaction studies, such as identification of receptor domains involved in ligand binding by production of immunoadhesins with systematically truncated receptor domains (Chamow and Ashkenazi (1996) TIBTECH Vol 14). In addition, new proteins capable of binding immune suppressive factors may be identified by the two-hybrid system protein interaction trap (Ausubel, et al. (1995) Short Protocols in Molecular Biology, 3rd Ed., Wiley). The DNA sequence encoding any such ligand binding domain may be ligated to a signal sequence for extracellular secretion and engineered into a suitable vector for gene delivery.

Another embodiment of the invention embraces fusion of a DNA sequence encoding a binding site for an immune suppressive factor, such as the extracellular domain of a receptor for an immune suppressive factor, to a DNA sequence encoding an immunoglobulin heavy chain backbone to produce an immunoadhesin. Immunoadhesins are antibody-like molecules that combine framework sequences from monoclonal antibodies with proteins that carry ligand binding functions. Like antibodies, immunoadhesins can be classified into different isotypes, depending on the immunoglobulin backbone from which they are constructed. There are five immunoglobulin backbone isotypes differing in the structure of their heavy chains: IgM, IgD, IgG, IgA and IgE. An immunoadhesin may combine the hinge and Fc regions of an immunoglobulin, such as an IgG or IgA heavy chain with domains of a cell surface receptor that recognizes a specific ligand. A typical immunoadhesin is a disulfide-linked homodimer resembling an IgG molecule but lacking C_{H}1 domains and light chains. Alternatively, an immunoadhesin may be constructed from an IgM backbone, leading to a multimeric immunoadhesin that may bind with increased avidity to its target. In certain embodiments, an immunoadhesin is constructed as an immunoadhesin-monoclonal antibody hybrid molecule. Such a hybrid immunoadhesin comprises an immunoadhesin chain and an antibody heavy and light chain pair which may or may not be bispecific for two different target molecules. Any of the foregoing immunoadhesins can be engineered into a vector disclosed herein and administered with a vaccine, resulting in the expression and secretion of the neutralizing immunoadhesin thereby stimulating an immune response to the vaccine.

Within another preferred embodiment, a neutralizing construct composed of both the heavy and light chains of a monoclonal antibody capable of neutralizing an immune suppressive factor is engineered. This entails the construction of a dual-gene delivery vector capable of directing the expression of a functional antibody specific for an immune suppressive factor in order to eliminate the need for co-delivery of separate vectors to a targeted cell. The dual-gene delivery vector is adapted for the expression and secretion of functional homo- or heterodimeric proteins from a single cell to which the vector has been delivered. Thus, the dual-gene delivery vector increases the effective concentration of a homodimer and obviates the need for separate expression of the protein subunits from different constructs and for multiple injections or other delivery modes with separate vectors. Alternatively, separate gene delivery vectors encoding the heavy and light chains of the antibody can be used for co-delivery into targeted cells. Administration of the dual-gene delivery vector leads to the expression and secretion of functional antibody or homodimer by transfected cells, causing the neutralization and blocking of the immune suppressive factors present systemically or in the tumor microenvironment.

All of the above constructs can be engineered into a suitable gene delivery vector used for transfer to cells, resulting in the production and secretion of neutralizing activity against immune suppressive factors and allowing the generation of an effective immune response directed against a vaccine antigen.

In another preferred embodiment, the DNA sequence encoding a neutralizing factor is engineered into a vaccinia recombination plasmid used to produce recombinant vaccinia vector for delivery of the neutralizing factors. Vaccinia, a double stranded DNA poxvirus, can be engineered to deliver up to 25 kb of heterologous DNA to a wide variety of mammalian cell types. The virus remains in the cytoplasm of the infected cells and uses virally encoded polymerases to carry out replication and transcription. The vaccinia infectious cycle consists of three phases: early, intermediate and late. During the early phase, genes encoding proteins with enzymatic function are expressed before replication. After viral replication is initiated, expression of intermediate genes drives the expression of structural proteins and other products of the late genes. Vaccinia late gene promoters are generally stronger than early promoters, making the late promoters suitable for high-level gene expression.

Recombinant vaccinia virus vectors encoding heterologous DNA can be generated by site-specific recombination with plasmids into which a gene or genes of interest have been inserted. Recombination plasmids contain a vaccinia virus promoter and two segments of the vaccinia virus genome flanking the promoter and inserted gene. The typical recombination site used is the viral thymidine kinase gene, which is disrupted by the recombination event. Recombination can be achieved by infecting cells, such as CV-1 cells derived from the African green monkey, with the wild-type virus, followed by transfection of the infected cells with the recombination plasmid. Alternatively, heterologous DNA up to a size of 25 kb can be ligated directly into the vaccinia genome, thus obviating the need for recombination and the associated procedures. Vaccinia has a high efficiency of infection or transfection and a broad host cell tropism allowing it to be targeted to multiple tumor types and neighboring tissues. Furthermore, vaccinia vectors confer high levels of gene expression even after multiple injections that provoke strong humoral responses to viral antigens.

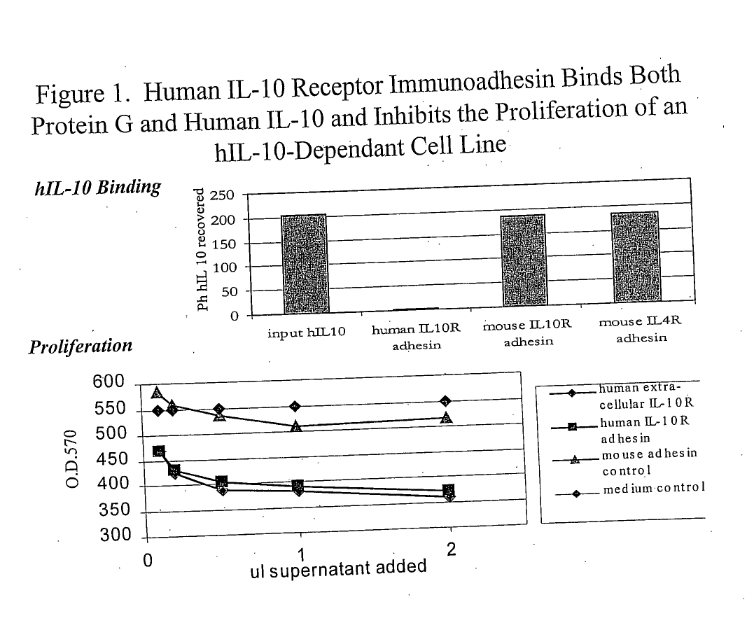

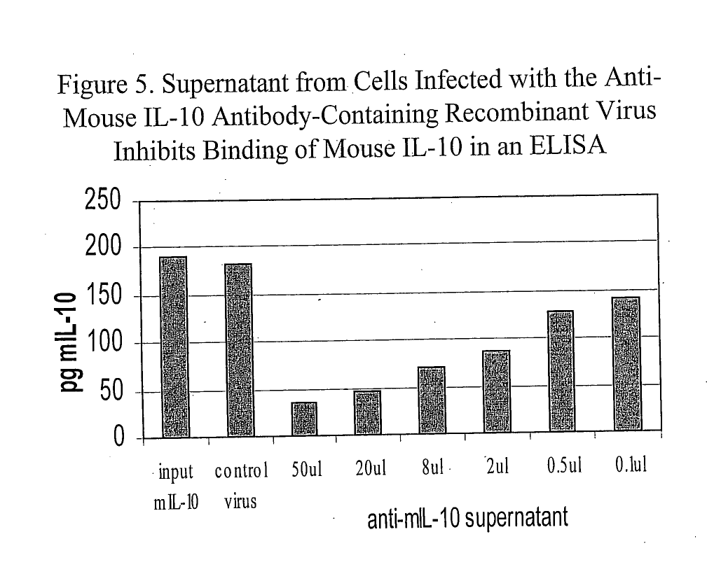

It has been demonstrated that the neutralizing constructs encoded by the recombinant vaccinia virus vectors inhibit immune suppressive factors associated with a tumor. As shown in FIG. 1 (bottom), the receptor extracellular domain binds the immune suppressive cytokine IL-10 and prevents it from stimulating the growth of an IL-10 responsive cell line. The IL-10 receptor immunoadhesin construct (FIG. 1, top) binds IL-10 and removes it from the media. Likewise, FIG. 5 demonstrates that the antibody construct specific for IL-10 is able to effectively prevent the binding of IL-10 in an ELISA. Moreover, it has been shown that a dual-gene delivery recombinant vaccinia vector encoding a neutralizing anti-IL10 antibody fusion molecule exhibits adjuvant activity when administered in combination with a vaccinia vector encoding the Uty tumor antigen (FIG. 13). As such, the vectors of the present invention are particularly suitable for use in combination with a variety of traditional vaccines to infectious diseases as well as tumor cell-based vaccines, tumor peptide vaccines, or polynucleotides coding for tumor antigens, as a second indication to enhance vaccination and to overcome evasion of the immune system.

Other vaccines suitable for combination with the present invention include wild-type vaccinia (U.S. Pat. No. 6,177,076), recombinant vaccinia encoding immune stimulatory cytokines such as GM-CSF, IL-12, or irradiated autologous tumor cells engineered to express any of the immune stimulatory cytokines. Such vaccine formulations may comprise one or more traditional adjuvants. An adjuvant is a substance that may be added to a therapeutic or prophylactic agent such as a vaccine or an antigen used for immunization in order to stimulate the immune response. Adjuvants and their use in vaccines to enhance immune responses are well-known in the art. In addition, the neutralizing strategy of the present invention can be used in combination with any suitable gene delivery method that introduces immune stimulatory factors into the host. Such a combination treatment system may lead to synergies in producing the regression of tumors by first eradicating the immune suppressive environment maintained by the tumor and subsequently potentiating the immune system attack by use of the pro-immune cytokines.

For example, vaccinia virus engineered to produce immune helper factors such as GM-CSF injected into tumors has been shown to result in enhanced antitumor effects and tumor regression (U.S. Pat. No. 6,093,700). This approach has been taken to clinical implementation and demonstrated significant clinical responses. To supplement and further augment the anti-tumor responses observed in the immunotherapy of cancer with immunostimulatory factors, gene delivery vectors engineered to produce neutralizing factors targeted against immune suppressive molecules present systemically or in the local tumor environment can be injected or otherwise delivered to the tumor site in conjunction with the immunostimulatory treatment. The vectors are designed to disrupt the local immune privilege created by the tumor-associated suppressive factors, thereby rendering the host capable of generating a productive antitumor immune response.

In one embodiment, the human IL-10 receptor immunoadhesin constructs are engineered from clone pSW8.1 (GenBank Accession #U00672) which contains the full-length human IL-10 receptor cDNA and is used as a template for the PCR amplification of the receptor extracellular domain sequence. This extracellular domain sequence is then ligated to the DNA sequences encoding the hinge, C_{H}2, and C_{H}3 domains of both human IgG1 and of human IgA1 to form anti-IL-10 immunoadhesins. The immunoadhesin cDNA is placed into the vaccinia recombination vector pSC65 (GenBank Accession #AX003206) and the plasmid is used to generate recombinant viruses. The ability of the neutralizing constructs to specifically bind to the immune suppressive factor can be demonstrated in the supernatants from cells infected with recombinant gene delivery virus.

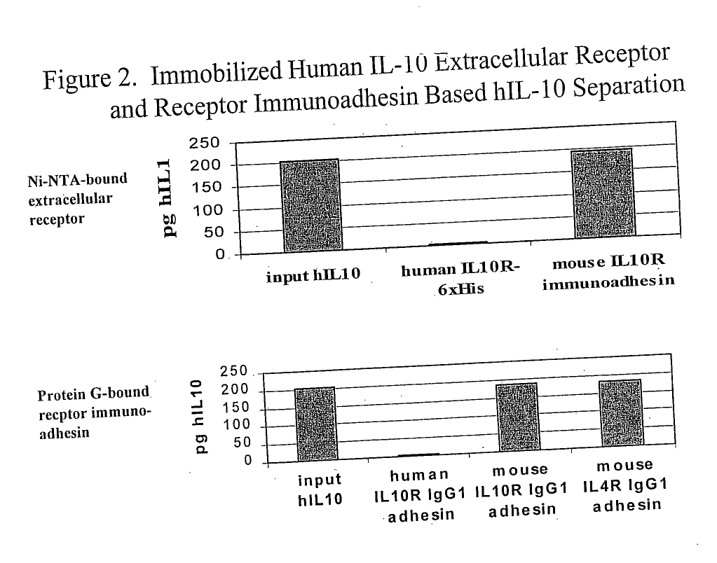

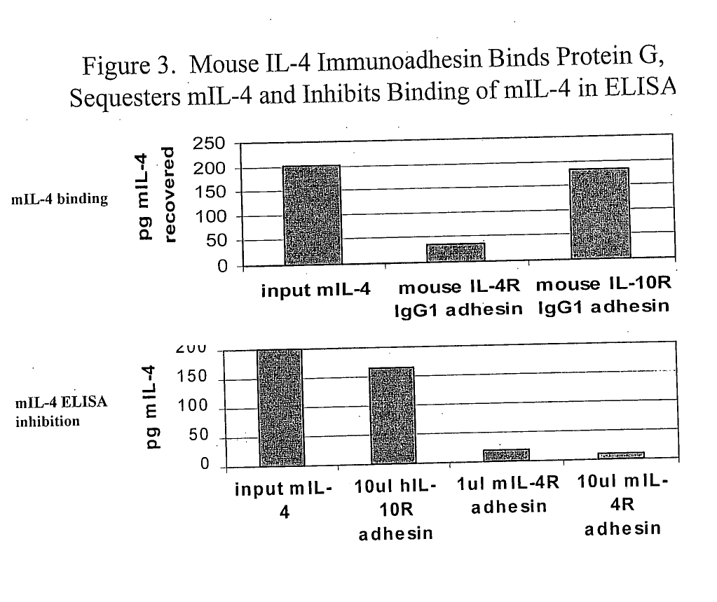

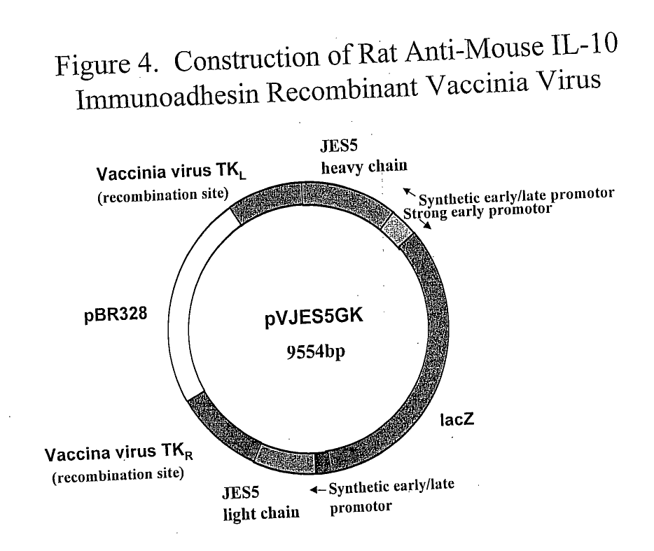

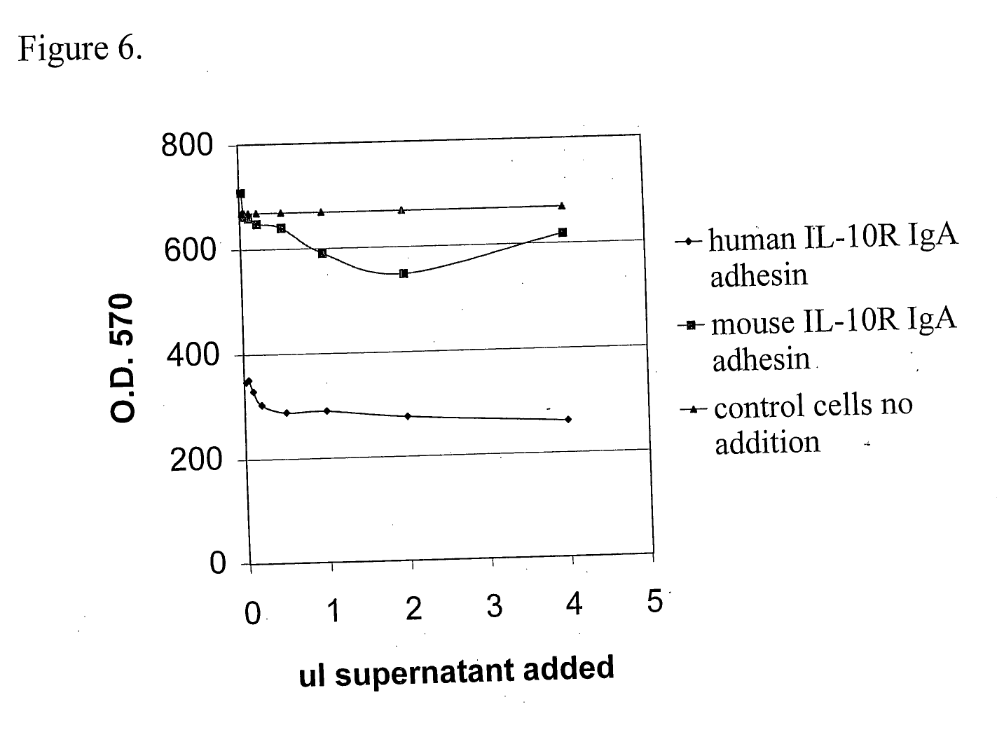

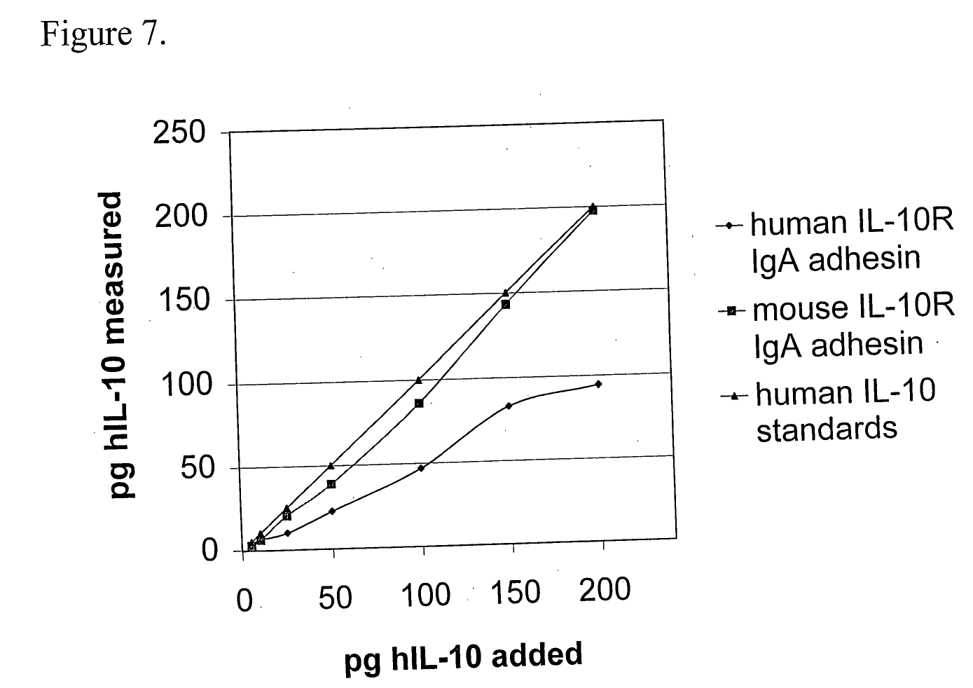

FIG. 1 (bottom) demonstrates strong and specific binding of the human IL-10 receptor IgG immunoadhesin to human IL-10 as evidenced by the low recovery of unbound hIL-10. This stands in contrast to the almost complete recovery of unbound human IL-10 when added to the murine IL-10 and IL-4 immunoadhesins. Further, FIG. 1 (bottom), shows that the extracellular domain of the human IL-10 receptor and the human IL-10 receptor IgG adhesin bind human IL-10 and inhibit IL-10 responsive proliferation of the cell line Ba8.1 as measured by the MTT assay. In addition, the immobilized extracellular domain of the receptor (human IL-10 R-6xHis) specifically binds and removes human IL-10 from media, as shown in FIG. 2, (upper panel) by the lack of recovery of input IL-10. Specificity of binding is shown by comparison with murine IL-10 receptor immunoadhesin (full recovery of added hIL-10). FIG. 3 (top) further shows specific binding of murine IL-4 receptor immunoadhesin to mIL-4, whereas murine IL-10 receptor immunoadhesin fails to bind to mIL-4. Similarly, murine IL-4 receptor immunoadhesin specifically binds mIL-4 and inhibits its activity in ELISA, as FIG. 3 (bottom) shows. Here, specificity is shown by comparison to the human IL-10 receptor immunoadhesin, which is unable to interfere with murine IL-4 receptor binding in ELISA. FIG. 5 shows the activity of the monoclonal antibody construct in the supernatant of cells infected with the gene delivery virus. The antibody inhibits binding of murine IL-10 in ELISA in a dose-dependent fashion. FIG. 6 demonstrates the strong and specific binding of human IL-10 receptor immunoadhesin with an IgA backbone to human IL-10, thus preventing the IL-10 from stimulating the proliferation of the IL-10 responsive Ba8.1 cell line. Specificity is shown in comparison to murine IL-10 receptor IgA immunoadhesin which has a negligible effect on IL-10 responsive cell proliferation in the same assay. Finally, the human IL-10 receptor IgA immunoadhesin specifically interferes with IL-10 activity in ELISA, as shown in FIG. 7 in comparison with Murine IL-10 receptor IgA immunoadhesin (no effect).

As used herein, “immune suppressive factor” denotes a molecule or compound which attenuates or inhibits one or more pathways of immune cell activation, proliferation or development. Examples of immune suppressive factors include IL-4, IL-10, VEGF, TGF-β, and prostaglandins.

A “cytokine” is a cell-derived soluble protein or peptide which acts as an immune regulator and modulates the functional activities of individual target cells and tissues. Cytokines are pleiotropic molecules, i.e., they can act as immune stimulants, immune suppressors, or immune modulators, capable of exerting their effects locally, systemically, or both. They can be subdivided into three (overlapping) functional categories: mediators and regulators of innate immunity, of adaptive immunity, and stimulators of hematopoiesis. Cytokines of innate immunity include TNF, IL-1, IL-12, IFN-α, IFN-β, IL-10, IL-6, IL-15, and IL18. Cytokines of adaptive immunity include IL-2, IL-4, IL-5, IFN-γ, TGF-β, lymphotoxin and IL-13. Cytokines that stimulate hematopoiesis include stem cell factor (c-Kit ligand), IL-7, IL-3, GM-CSF, IL-9 and IL-11.

“Prostaglandins” are lipid-soluble members of a family of 20 carbon containing hormones known as eicosanoids that are synthesized from a common precursor, arachidonic acid. Prostaglandins bind to cell-surface receptors and can exert profound effects on many cellular processes. PGD₂, for example, promotes neutrophil chemotaxis, while PGE₂ inhibits T-cell activation and mitogen-stimulated T-cell proliferation. Cyclopentenone prostaglandins may be overproduced in certain cancers and can impair the function of the immune system.

A “binding site”, “binding domain” or “ligand binding domain” is that region of a protein that associates with a ligand, which can be either another protein, DNA, hormone, or other compound.

A “soluble binding site” or “soluble receptor” refers to the binding site of a protein, often a membrane bound receptor, that has been separated from the membrane bound or otherwise non-soluble remainder of the protein.

“Neutralization” refers to the interaction of a compound including a molecule, such as a macromolecule, with an immune suppressive factor, such that the immune suppressive factor is prevented from interacting with its receptor and its activity is blocked.

The terms “neutralizing factor” or “neutralizing polypeptide” mean a recombinant polypeptide which binds a immune suppressive factor, thereby making the immune suppressive factor unavailable for interaction with other binding partners.

A “polypeptide”, as used herein, denotes a protein, thus referring to both a single chain polymer of amino acids connected by peptide bonds, as well as to a plurality of single chain amino acid polymers which can assemble into a protein composed of more than one identical or different subunits.

The term “neutralizing construct” refers to both the recombinant polypeptide which binds an immune suppressive factor, as well as to the polynucleotide sequence or sequences encoding the neutralizing polypeptide.

A “nucleic acid construct” or “DNA construct” is used to refer to a coding sequence or sequences operably linked to appropriate regulatory sequences that can be inserted into a vector for transforming a cell.

A cell has been “transformed” or “transfected” or “transduced” by exogenous or heterologous DNA when such DNA has been introduced inside the cell. The transforming DNA may or may not be integrated (covalently linked) into the genome of the cell. In prokaryotes, yeast and mammalian cells, for example, the transforming DNA may be maintained on an episomal element such as a plasmid.

A “vector” denotes a replicon, such as a plasmid, phage, cosmid, or virus into which heterologous nucleic acid segments may be operably inserted, capable of directing the expression or replication of such sequences or genes of interest in a host cell.

A “heterologous” region of a nucleic acid construct is an identifiable segment (or segments) of the nucleic acid molecule contained within or linked to another nucleic acid molecule with which it is not found associated in nature. Thus, when the heterologous region encodes a mammalian gene, the gene will usually be flanked by DNA that does not flank the mammalian genomic DNA in the genome of the source organism. In another example, a heterologous region is a construct where the coding sequence itself is not found in nature (e.g., a cDNA of a corresponding genomic coding sequence containing introns, or synthetic sequences having codons different from the native gene). Allelic variations or naturally occurring muational events do not give rise to a heterologous region of DNA as defined herein.

A “coding sequence” or coding region refers to a nucleic acid molecule having sequence information necessary to produce a gene product when the sequence is expressed.

The term “operably linked” means that the regulatory sequences necessary for expression of the coding sequence are placed in a nucleic acid molecule in the appropriate positions relative to the coding sequence so as to enable expression of the coding sequence. The same definition is sometimes applied to the arrangement of transcription control elements other than promoters (e.g. enhancers) in a gene delivery vector.

Transcriptional and translational control sequences are DNA regulatory sequences, such as promoters, enhancers, polyadenylation signals, terminators, and the like, that provide for the expression of a coding sequence in a host cell.

The terms “promoter”, “promoter region” or “promoter sequence” refer generally to transcriptional regulatory regions of a gene, which may be found at the 5′ or 3′ side of the coding region, or within the coding region, or within introns. Typically, a promoter is a DNA regulatory region capable of binding RNA polymerase in a cell and initiating transcription of a downstream (3′ direction” coding sequence). The typical 5′ promoter sequence is bounded at its 3′ terminus by the transcription initiation site and extends upstream (5′ direction) to include the minimum number of bases or elements necessary to initiate transcription at levels detectable above background. Within the promoter sequence is a transcription initiation site (conveniently defined by mapping with nuclease S1), as well as protein binding domains (consensus sequences) responsible for the binding of RNA polymerase.

By “immunoadhesin” is meant a chimeric molecule composed of a non-immunoglobulin binding region, such as that of a receptor, cell adhesion molecule, enzyme, or ligand, and an antibody heavy chain hinge and constant region.

The term “therapeutically effective amount” denotes an amount of a compound, such as a gene delivery vector carrying the coding sequence for a particular therapeutic construct, such that when the compound is administered to a subject it is effective to bring about a desired effect (e.g., neutralization of the immune suppressive factors) within the subject.

In some embodiments of the invention, neutralizing constructs are used in combination with vaccines for infectious diseases such as hepatitis, small pox, measles, mumps, etc. In particular embodiments, the neutralizing constructs are used in the immunotherapeutic treatment of tumors. Primary or metastatic tumors can be injected with a vector for the delivery of the neutralizing constructs, such as recombinant virus encoding any of the neutralizing constructs, resulting in the production of the neutralizing constructs by the tumor cells or cells in the tumor environment. The neutralizing constructs are secreted into the tumor environment and in turn bind and inhibit the tumor-associated suppressive molecules. Similarly, bladder cancer can be treated locally by the introduction of a vector, such as a recombinant virus encoding the neutralizing constructs, into the bladder with a catheter. The development of antitumor immunity could be further enhanced by combination with pro-immune cytokine constructs. Because the neutralizing constructs are also useful as adjuncts to tumor antigen encoding vaccines, vectors encoding the neutralizing constructs could be administered in conjunction with the administration of vectors encoding pro-immune cytokines to enhance an effective immune reaction specific for a tumor thereby preventing tumor development.

In another preferred embodiment of the invention, the vectors encoding neutralizing activity for the immune suppressive factors, such as IL-10, IL-4, VEGF, TGF-β and prostaglandins as examples, which are normally produced in the process of an immune response and which suppress the development of cell-mediated immunity, are used as adjuncts to traditional vaccines aimed at enhancing the development of cell-mediated immune responses.

The compositions and methods of the present invention can be used for any host. Preferably, the host will be a mammal. Preferred mammals include primates such as humans and chimpanzees, domestic animals such as horses, cows, pigs, dogs, and cats. More preferably, the host animal is a primate or domestic animal. Still more preferably, the host animal is a primate such as a human. The compositions and methods of the invention are also suitable for the treatment of a variety of solid tumors and their metastases, regardless of their location and origin. Thus, for example, cancers of the breast, colon, esophagus, bile duct, gallbladder, liver, pancreas, rectum, small intestine, stomach, thyroid, bladder, kidney, prostate, testes, urethra, cervix, endometrium, ovaries, uterus, vagina, vulva, head and neck (hypophanryngeal, laryngeal, lip and oral cavity, nasopharyngeal, oropharyngeal, paranasal sinus and nasal cavity, parathyroid and salivary gland), lung, mesothelium, muscle (rhabdomyosarcoma, soft tissue sarcoma, uterine sarcoma), skin (melanoma, Kaposi's sarcoma, skin cancer, Merkel cell carcinoma), hematologic cancers manifesting as solid tumor masses (cutaneous T-Cell lymphoma as an example), as well as their metastases, including those of unknown primary tumors, are suitable for treatment using the compositions and methods of the present invention.

The vectors may be administered in an amount that results in measurable expression of the neutralizing factors and an enhanced immune response to an antigen. A person of ordinary skill in the art is able to routinely determine what that amount is. Dosage levels and frequencies of administration of other delivery vectors are routinely determined by those skilled in the art.

In a preferred embodiment, the compositions of the invention are formulated for inclusion into a kit for administration to a subject. The kit contains therapeutically effective amounts of the formulated gene delivery vectors for the expression of one or more of the neutralizing factor and can further comprise an antigen-encoding vaccine. Such a kit may also contain a set of instructions for the application of the formulated gene delivery vectors in vivo.

The following examples are provided to better illustrate the claimed invention and are not to be interpreted as limiting the scope of the invention. To the extent that specific materials are mentioned, it is merely for purposes of illustration and is not intended to limit the invention. Unless otherwise specified, general cloning procedures, such as those set forth in Sambrook et al. ((2001) Molecular Cloning, Cold Spring Harbor Laboratory; Ausubel, et al. (2000) supra are used. One skilled in the art may develop equivalent means or reactants without the exercise of inventive capacity and without departing from the scope of the invention.

It will be understood that many variations can be made in the procedures herein described while still remaining within the bounds of the present invention. Likewise, it is understood that, due to the degeneracy of the genetic code, nucleic acid sequences with codons equivalent to those disclosed will encode functionally equivalent or identical proteins as disclosed herein. It is the intention of the inventors that such variations are included within the scope of the invention.

### EXAMPLE 1

### Recombinant Vaccinia Encoding the Extracellular Domain of IL-10 Receptor

This example illustrates the construction of a gene delivery vector capable of directing the expression of the extracellular domain of a receptor for an immune suppressive factor. Recombinant Vaccinia producing the human IL-10 receptor extracellular domain is generated using standard techniques. The wild-type (wt) virus used in the preparation of the recombinant is derived from the Wyeth strain of vaccinia (Centers for Disease Control, Atlanta, Ga.). The full-length cDNA for human IL-10 receptor is obtained from clone pSW8.1 (GenBank Accession #U00672) and the extracellular domain is PCR amplified using the h10RKCS plus strand primer (SEQ ID NO:1) and the h10R6H minus strand primer (SEQ ID NO:2). The DNA sequence encoding the IL-10 receptor extracellular domain is cloned first into the HindIII/BamHI site of pBLUESCRIPT II SK (STRATAGENE, La Jolla, Calif.) and subsequently into the Sal1 and Not1 sites of pSC65 (GenBank Accession #AX003206). This plasmid is then transfected with LIPOFECTIN (GIBCO-BRL) into CV-1 monkey kidney cells that have been infected two hours previously with a low multiplicity (0.05-0.1) of wild type virus. The plasmid is designed to facilitate homologous recombination into the vaccinia thymidine kinase (TK) gene. Recombinants are selected in 143B (human osteosarcoma, TK negative) cells in the presence of 5-bromo-2′-deoxyuridine. The lacZ gene is included in the plasmid as a reporter gene. Following three rounds of selection, the virus is plaqued to confirm that a pure recombinant stock has been obtained. The presence of the DNA sequence encoding the IL-10 receptor extracellular domain in virus is confirmed by PCR.

### EXAMPLE 2

### Construction of Recombinant Vaccinia Encoding IL-10-R Immunoadhesins

This example illustrates the construction of an immunoadhesin capable of neutralizing an immune suppressive factor. The DNA sequence encoding the extracellular domain of a receptor for an immune suppressive factor is isolated as described in Example 1 and ligated to a DNA sequence coding for an immunoglobulin backbone. The resulting chimeric DNA sequence coding for the immunoadhesin is subcloned into an expression vector. Clone pSW8.1 (GenBank Accession #U00672) containing the full-length human IL-10 receptor cDNA is used as a template for the PCR amplification of the receptor extracellular domain sequence (SEQ ID NO:3). The IL-10 sequence is PCR amplified by the use of the h10RIA3 minus strand primer (SEQ ID NO:4) which includes a Sal1 site for ligation of the hIL-10 extracellular receptor domain DNA sequence to the DNA sequences of the human IgG1 and IgA1 hinge region domains; and the h10RKCS plus strand primer (SEQ ID NO:1) which includes a HindIII site for cloning and a Kozak concensus sequence. The IgA and IgG immunoglobulin backbone sequences are PCR amplified from cell line DAKIKI (ATCC #TIB-206) and ARH-77 cDNA (ATCC #CRL-1621), respectively. For the IgA backbone amplification, the higAM minus strand primer (SEQ ID NO:5) for the human IgA1 hinge, C_{H}2, and C_{H}3 domains which includes a BamHI site for cloning, and the higAP2 plus strand primer (SEQ ID NO:6) which includes a SalI site for cloning and attachment of binding domains are used. For the amplification of the IgG backbone containing the human IgG1 hinge, CH², and CH3 domains, the higG1P plus strand primer (SEQ ID NO:7) which includes a SalI site for cloning and ligation of the DNA sequences of the binding domains and the higGM minus strand primer (SEQ ID NO:8) which includes a BamHI site for cloning are used.

The amplified extracellular domain sequence of the IL-10 receptor is ligated to the amplified DNA sequences of the hinge, C_{H}2, and C_{H}³ domains of human IgG1 and of human IgA1 to form the DNA sequences of anti-IL-10 immunoadhesins. The immunoadhesin DNA sequences are subcloned into the vaccinia recombination vector pSC65 (GenBank Accession #AX003206) and the plasmid is used to generate recombinant virus.

### EXAMPLE 3

### Construction of a Dual-Gene Vaccinia Recombination Plasmid

This example illustrates the construction of a vector specifically adapted for the expression of two genes in the production and secretion of functional engineered antibodies and other heterodimeric proteins as well as both subunits of a homodimer by the same infected cell. For this purpose, a vaccinia recombination plasmid containing the following elements is synthesized: 1) two strong early/late vaccinia promoters for high level transcription, 2) vaccinia thymidine kinase (TK) sequences for homologous recombination with the TK locus of the viral genome allowing easy selection of TK negative recombinants, and 3) the E. coli lacZ gene useful for identifying recombinant viruses and for staining of tissue samples to demonstrate viral infection and replication.

Two of the single-gene vaccinia recombination plasmids that are suitable for construction of a dual-vector are pSC11/9 and pSC65 (GenBank Accession #AX003206). Both vectors contain the lacZ gene and use TK sequences for recombination. To regulate lacZ transcription pSC11/9 uses the strong p11 late promoter while pSC65 uses the moderately active p7.5 early/late promoter. The pSC11/9 multiple cloning site (MCS) has seven unique restriction endonuclease sites: SalI, AflII, SacII, NheI, ApaI, KpnI, NotI; the pSC65 MCS has 6 unique restriction sites: SalI, BglII, StuI, KpnI, NotI, PacI. Transcription of genes inserted into the pSC11/9 MCS is regulated by the p7.5 early/late promoter while pSC65 uses a strong synthetic early/late promoter.

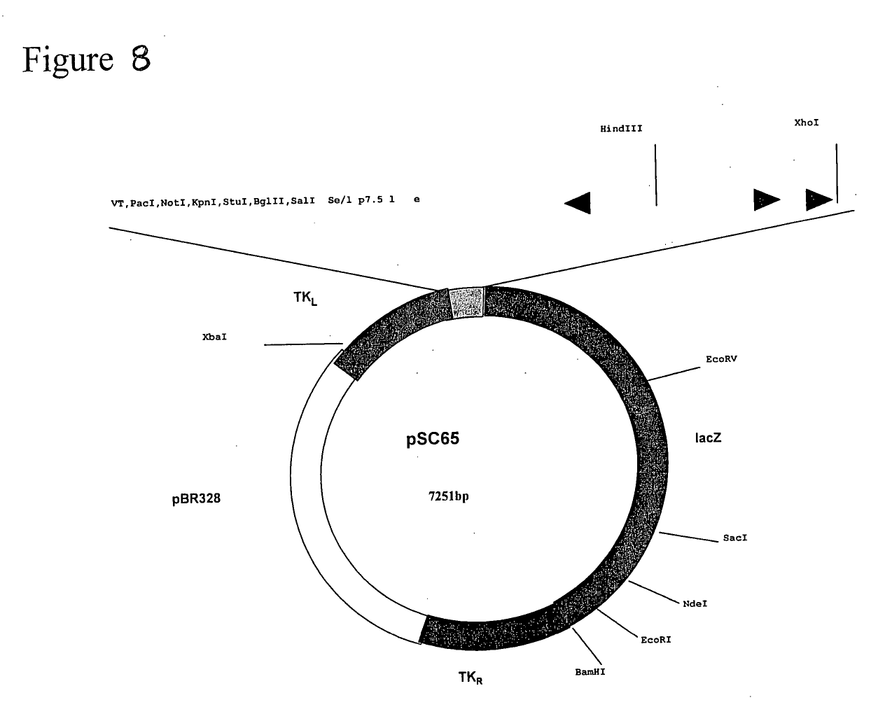

pSC65 (GenBank Accession #AX003206) is used for construction of the dual vector because of its convenient single restriction endonuclease sites (FIG. 8) which facilitate manipulation of the plasmid. The synthetic early/late promoter from pSC65 is selected to regulate transcripton in the dual-vector. This promoter produces a high level of transcription during both the early phase and the late phase of vaccinia replication and has demonstrated success in the pSC65 background with the vaccinia-GM-CSF recombinant virus currently in clinical trials.

Converting pSC65 (GenBank Accession #AX003206) into a dual-vector requires the addition of a second promoter along with an associated multiple cloning site. There are two possible locations for a second promoter/MCS in pSC65: (1) back-to-back with the synthetic early/late promoter/MCS or (2) in the same orientation as and following the lacZ gene. The latter position is suitable for the second promoter because it generates a more stable plasmid; the back-to-back arrangement of two identical promoters creates an inverted repeat sequence which is susceptible to DNA rearrangement.

Because of the location of the second promoter/MCS in the same direction as and following the lacZ gene it is necessary to modify the p7.5 early/late promoter to prevent the upstream promoter from affecting transcription from the downstream second promoter. One modification of the p7.5 promoter involves removing the late promoter sequences. This alteration, however, leaves only the p7.5 early promoter to regulate lacZ transcription. pSC11/9 and pSC65 both use late promoters to regulate lacZ transcription and, as experience has shown, produce adequate levels of β-galactosidase activity for plaque identification and tissue staining. The p7.5 early promoter is much weaker than both the p11 late promoter and the p7.5 early/late promoter. Therefore, the p7.5 early promoter sequence is modified to increase its strength and to compensate for the loss of late promoter activity. Four single-base changes in the critical region of the p7.5 early promoter increase lacZ expression four-fold and produce adequate levels of β-galactosidase activity for plaque identification of dual-vector recombinant viruses.

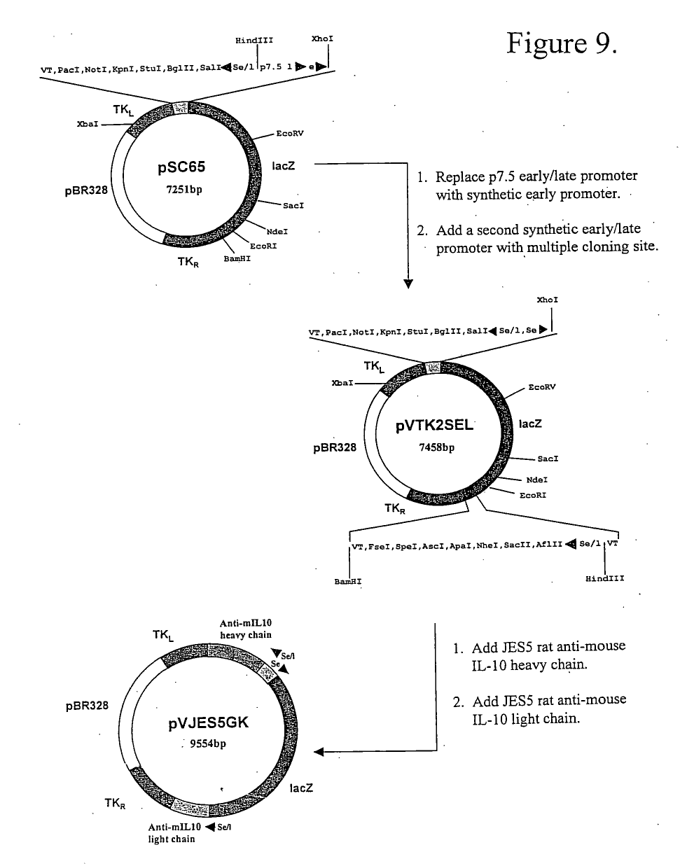

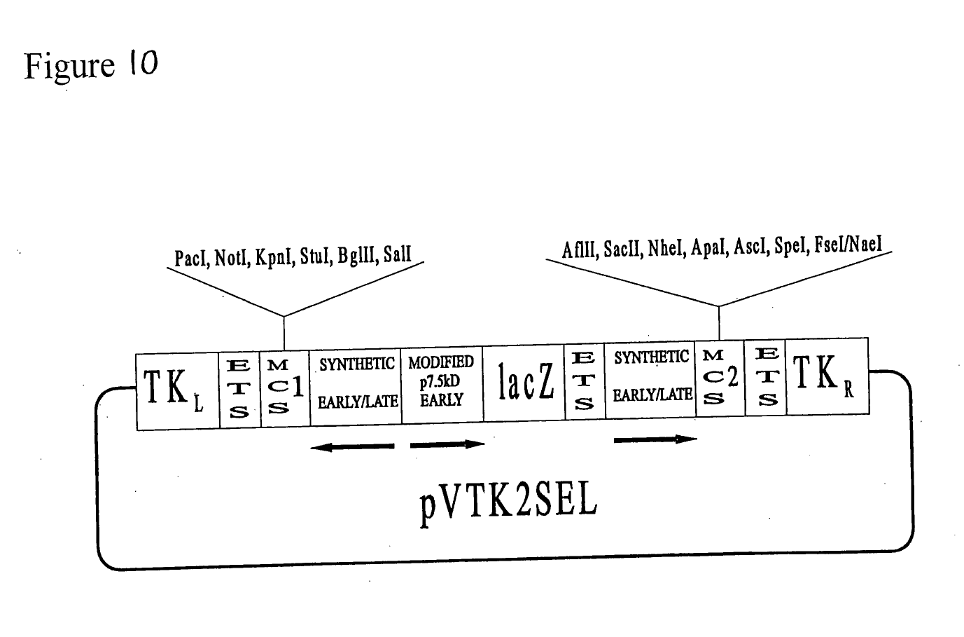

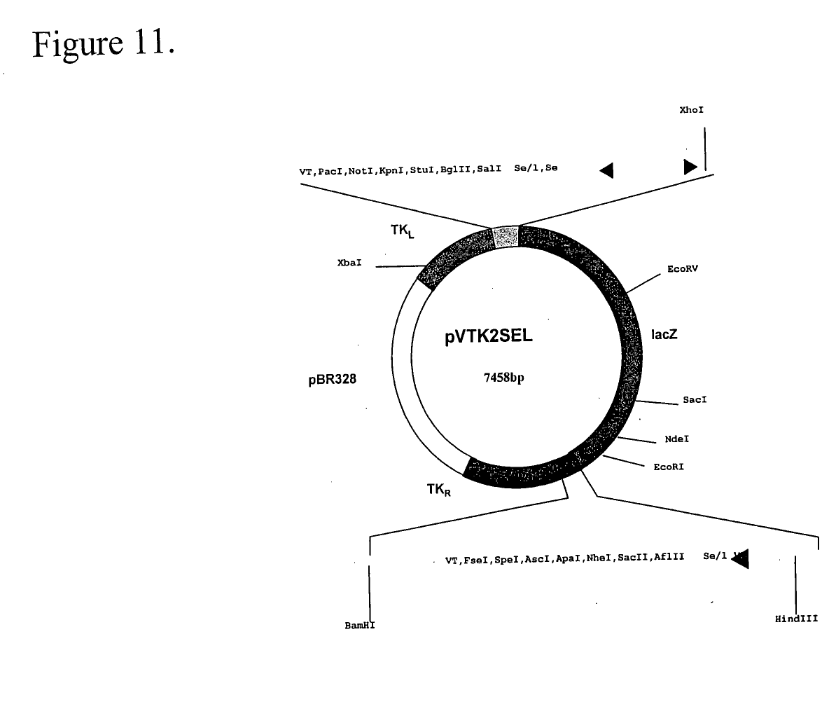

The second promoter/MCS is inserted into pSC65 at the single BamHI site, located between the end of the lacZ gene and the start of the right-hand TK sequences (FIG. 9). This is accomplished by first isolating the SacI/BamHI fragment of pSC65 and subcloning it into the plasmid BLUESCRIPT. At the BamHI site of this construct four elements are added: an early transcription termination signal (TTTTTAT; SEQ ID NO:9), the synthetic early/late promoter from pSC65, a modified multiple cloning site from pSC11/9, and a second early transcription termination signal. The MCS from pSC11/9 is modified by eliminating the SalI and KpnI restriction endonuclease sites, converting the NotI restriction site into a FseI/NgoMI site, and adding AscI and SpeI restriction sites; the second MCS contains the following restriction endonuclease sites: AflII, SacII, NheI, ApaI, AscI, SpeI, FseI/NgoMI. The modified SacI/BamHI fragment, now including the second promoter/MCS, is excised from BLUESCRIPT and ligated into a pSC65 fragment containing the p7.5 promoter modifications outlined above to give the dual-vector pVTK2SEL (FIGS. 10 and 11).

### EXAMPLE 4

### Preparation of Vaccinia Virus Expressing Functional Antibody

This example illustrates the construction of a DNA sequence encoding a synthetic monoclonal antibody specific for an immune suppressive factor, followed by the expression of the synthetic antibody from cells infected with recombinant vaccinia virus encoding the DNA sequence for the antibody. An antibody-based anti-IL-10 construct using the heavy and light chains of an anti-murine IL-10 monoclonal antibody JES5 is constructed for use in neutralizing IL-10 as follows: the rat anti-mouse IL-10 antibody is cloned from hybridoma JES5. JES5 RNA is reverse transcribed using primers specific for the constant region of the rat kappa chain, Rat K1 (SEQ ID NO:10), and for the rat gamma chain C_{H}2 domain, Rat G1 (SEQ ID NO:11). A 5′ dG tail is added by terminal deoxynucleotidyl transferase to each cDNA which are then PCR-amplified using a specific minus-strand nested primer, Rat G2 (SEQ ID NO:12) for the gamma chain and Rat K2 (SEQ ID NO:13) for the kappa chain, and a common oligo dC primer, 3 GT (SEQ ID NO:14) containing several restriction sites to allow directional cloning of the products into BLUESCRIPT. Sequencing of both chains identified their 5′ start sites and leader sequences and their uniqueness indicated that they are not the products of aberrant transcripts. The two genes are cloned into the newly designed dual vector as outlined below.

The polydG is removed from each chain by PCR using as plus strand primers RatG3 (SEQ ID NO:15) and RatK3 (SEQ ID NO:16) which include the first 24 residues of the gamma and kappa chains, respectively, and add a Kozak consensus sequence to the start site and a HindIII site for cloning into BLUESCRIPT, paired with minus-strand primers RatG2 (SEQ ID NO: 12) and RatK2 (SEQ ID NO:13). The DNA sequences coding for the V and C_{H}1 domains of the JES5 gamma chain are removed from BLUESCRIPT using BamHI and AvrII and ligated to the DNA sequences encoding the hinge, C_{H}2, and C_{H}3 domains of murine IgG1 before being cloned into the dual vector pVTK2SEL at Sal1/Not1. In the kappa chain BLUESCRIPT construct the HindIII site is first changed to a NheI site and the NotI site changed to a FseI/NgoMI site before the kappa chain can subsequently be cloned into the NheI/FseI site of the gamma chain construct in pVTK2SEL to give the vaccinia recombination plasmid pVSJES5GK containing both antibody genes (FIG. 9).

To generate a recombinant vaccinia virus capable of expressing a functional antibody, the Wyeth strain of vaccinia virus is used as the parental strain. The vaccinia virus Wyeth from the Centers for Disease Control and Prevention is the same virus contained in the smallpox vaccine which is currently administered as a precaution to laboratory personnel working with vaccinia virus.

The homologous recombination is performed in CV-1 cells that have been both inoculated with the Wyeth strain, at a multiplicity of infection of 0.05-0.1, and transfected with a mixture of 5-10 ug of pVJES5GK and LIPOFECTIN (GIBCO-BRL). Two days following this treatment the cells are harvested and a lysate made by several freeze-thaw cycles accompanied by sonication. The lysate is used to inoculate Human TK-negative 143B cells grown in the presence of bromodeoxyuridine; this step serves to expand the small number recombinant viruses initially produced by selecting for viruses with a disrupted thymidine kinase gene. A lysate made from the TK-negative cells is used in a first round of plaque purification where TK negative cells are inoculated with the lysate, two hours later overlaid with agarose, then two days later overlaid with a second layer of agarose containing X-gal. Several desirable recombinant viruses, those that produce large, dark blue plaques are picked and one is further plaque purified several, times before it is considered substantially homogenous and free of spontaneously-formed TK-negative viruses (those forming colorless plaques). The candidate recombinant virus is expanded, titered, and tested for functional IL-10 antibody production by ELISA (PHARMINGEN) and cell proliferation assay.

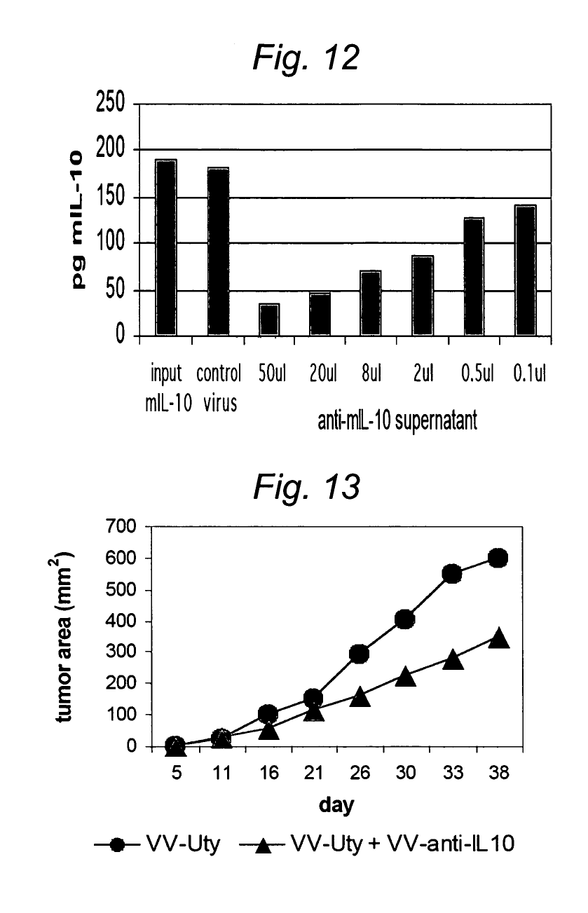

Using such an approach, a recombinant vaccinia encoding a neutralizing anti-IL10 antibody fusion molecule was produced. The construct was based on the heavy and light chains of an anti-murine IL10 monoclonal antibody JES5. The rat anti-mouse IL-10 antibody was cloned from hybridoma JES5. JES5 RNA was reverse transcribed using primers specific for the constant region of the rat kappa chain and for the rat gamma chain C_{H}2 domain. A 5′ dG tail was added to each cDNA which were then PCR-amplified using specific minus-strand nested primers and a common oligo dC primer containing several restriction sites to allow directional cloning of the products. Sequencing of both chains identified their 5′ start sites and leader sequences and their uniqueness indicated that they were not the products of aberrant transcripts. The two genes were cloned into a vaccinia dual-gene delivery vector as disclosed herein. Following generation and cloning of the recombinant virus, supernatant was harvested from infected TK-cells, concentrated, and mixed with a constant amount of mIL-10 before being assayed by ELISA. FIG. 12 demonstrates that the supernatant has significant levels of neutralizing activity.

To demonstrate that the anti-IL-10-producing vaccinia virus could have an adjuvant effect on vaccination against tumor antigen, mice were co-injected with the vaccinia virus expressing anti-IL10 antibody in combination with tumor antigen Uty. Two groups of eight mice were vaccinated in the right groin with a mixture of 3×10⁶ pfu of Uty-producing vaccinia along with 1×10⁶ pfu of GM-CSF-producing vaccinia. In the same injection, one group also received 9×10⁶ pfu of control virus producing only beta-galactosidase while the second group received 9×10⁶ pfu of anti-IL-10-producing vaccinia. Fifteen days later, all mice received a second dose of the same vaccination mixture. Nineteen days followed before all of the mice were challenged in the left groin with 1×10⁵ MB49 cells suspended in 100 ml PBS. Measurements of tumor size were taken every 3-5 days and recorded as two dimensions. Mice were terminated when tumor size exceeded 600 mm². Vaccinia virus expressing anti-IL10 enhanced the protective capability of vaccinia virus expressing Uty when given prior to tumor cell injection resulting in a slowing of tumor growth (p=0.01) (FIG. 13).

### EXAMPLE 5

### Binding Activity of the Neutralizing Constructs

This example illustrates that cells infected with the recombinant vaccinia vectors express and secrete the immunoadhesins and extracellular domains of the receptor for an immune suppressive factor. It also demonstrates that the extracellular receptor domains and immunoadhesins bind their respective ligands with specificity. In order to ascertain the binding activity of the newly constructed extracellular receptor binding domain and immunoadhesin constructs, a binding assay is designed as follows: 250 μl of beads (Protein G-SEPHAROSE (PHARMACIA); Ni-NTA-Agarose (QIAGEN)) are incubated with concentrated supernatant in a 1.5 ml centrifugal filter tube (MILLIPORE) for 30 minutes at room temperature. The beads are pelleted by spinning, the supernatant is removed and the beads rinsed 3× with 300 μl PBS/FBS by alternately resuspending and pelleting. 250 μl of 200 pg/100 μl of hIL-10 or mIL-4 are added and the mixture incubated for 30 minute at room temperature. After spinning down the beads, the filtrate is recovered and assayed for either hIL-10 or mIL-4 by ELISA (PHARMAGEN). Results: as shown in FIGS. 1 (top), 2 and 3 (top), the hIL-10 receptor ligand binding domain and the hIL-10 receptor immunoadhesins bind strongly and specifically to hIL-10, while the mIL-4 receptor immunoadhesin binds strongly and specifically to mIL-4.

### EXAMPLE 6

### ELISA Inhibition of the Neutralizing Constructs

This example illustrates that the extracellular domains, immunoadhesins and synthetic antibody produced and secreted by the vector-infected cells bind to their respective immune suppressive factors, thereby preventing the interaction of the immune suppressive factor with any other target. To determine the binding activity of the concentrated supernatants from cells infected with recombinant vaccinia encoding either hIL-10 R IgA immunoadhesin, mIL-4R IgG immunoadhesin, or mIL-10R antibody, different volumes of concentrated supernatants are incubated at room temperature for 30 min with 250 pg of either hIL-10, mIL-10, or mIL-4. Following the incubation period a volume equivalent to 200 pg of cytokine/supernatant mixture is removed and added to wells of a microtiter plate and assayed by ELISA (PHARMAGEN). The assay demonstrates strong and highly specific inhibition of target binding of the immune suppressive factors by the neutralizing constructs in ELISA. (FIGS. 3 (bottom), 5 and 7).

### EXAMPLE 7

### Neutralizing Activity of the Immunoadhesin and Binding Domain Constructs

This example illustrates the ability of the immunoadhesins and extracellular receptor domains to bind to the immune suppressive factors and remove them from the extracellular environment, thus making the immune suppressive factors unavailable for interaction with their cellular receptors. Ba8.1 cells (25,000 in 50 μl) are added to quadruplicate wells of a flat bottom 96-well cluster in RPMI medium containing 50 um β-mercaptoethanol, 2 ng/ml mIL-3, 10% FBS, and 0.5 mg/ml G418. Concentrated supernatants from virus-infected cells are appropriately diluted with the same medium, mixed with an equal volume of 400 pM hIL-10 and incubated at room temperature for 30 minutes to allow binding of the immunoadhesins and extracellular receptor domains to the hIL-10. Fifty μl of the extracellular receptor domain/hIL-10 or immunoadhesin/IL-10 mixture is added to each appropriate well containing Ba8.1 cells to give a final concentration of 100 pM of hIL-10 which is the optimum level to stimulate growth of the cells. After incubating at 37° C. for 48 hours, MTT ([3-(4,5-dimethylthiazol-2-yl)-2,5-diphenyl-2H-tetrazolium bromide, 2.5 mg/ml, 20 μl) is added to each well. Following an additional 4 hours at 37° C., the cells and reduced MTT are dissolved by the addition, with pipetting, of 100 μl of a mixture of acid/alcohol (20 ml of 1N HCl and 0.5 ml NP-40 in 500 ml n-propanol). The OD 570 nm is determined using a plate reader. Results: As shown in FIGS. 1 (bottom) and 6, the immunoadhesin and receptor ligand binding domain constructs strongly inhibit growth of the hIL-10 responsive murine cell line.

### EXAMPLE 8

### In Vivo Administration of Vector to Tumor Bearing Mice

This example illustrates the measurement of the effect of the expression and secretion of the neutralizing factors on tumor growth in vivo. Several mouse tumor models can be used to assess the effect of inhibiting tumor-associated local release of immune suppressive factors on the growth or regression of solid tumors and their metastases. In addition, the impact of delivery of the neutralizing factors on the effectiveness of a vaccine containing a relevant tumor antigen can be determined. Age-matched female C57BL/6 mice, obtained from The Jackson Laboratory (Bar Harbor, Me.) are injected subcutaneously (solid tumor model), intravenously in the tail vein (lung metastasis model) with 1×10⁶ T241 murine fibrosarcoma, B16-F10 melanoma, CT-26 colon carcinoma, LLC, or MB-49 bladder tumor cells. Seven to fourteen days following tumor development, the tumors are located visually or by X-ray, ultrasound, CT scan or other imaging methods known to those skilled in the art. The tumor-bearing mice are then injected with 1 to 2×10⁶ pfu recombinant vector expressing the neutralizing constructs either alone or in combination with a vaccine containing or encoding a relevant tumor antigen in a total of 100 μl. The injections can be repeated at different intervals and can be combined with injections of vector encoding pro-immune cytokines. The size of the solid tumors can be measured every 2-3 days with metric calipers by measuring the two largest diameters. For internal tumors, change in size can be monitored by standard imaging methods known to those skilled in the art.

### EXAMPLE 9

### Intralesional Introduction of Recombinant Vector into Human Tumors

Eligible tumor bearing patients are tested for immune competence with dinitrofluorobenzene. The dinitrofluorobenzene is prepared before each application by dissolution in acetone-to-corn oil (9:1). Sensitization is accomplished by topical application of 1.0 mg dinitrofluorobenzene to a skin site on the volar surface of the forearm in the confines of a 1 cm circle. Challenge consists of the topical application of 0.05, 0.10, and 0.20 mg to separate naive skin sites on the forearm. Delayed type hypersensitivity reactions are scored as positive if any of the concentrations produce a full circle of erythema and induration after 48 hours.

Patients who are immunocompetent are immunized with vaccinia virus using the standard multi puncture technique. Individuals with a major vaccinoid type skin reaction, which is usually discernible at 4 days after vaccination are eligible for treatment. On day 4, intralesional or intravesical therapy is begun and repeated twice or three times weekly with dose escalation based on the local (erythema, inflammation) or systemic (clinical symptoms, physical signs, and clinical laboratory values) toxicity form the preceding injections. Escalating doses of 10⁴ to 2×10⁷ pfu per lesion and 10⁴ to 10⁸ pfu per treatment session can be administered. Toxicity can be graded using the National Cancer Institute common toxicity criteria.

What is claimed is:

1. A vaccine adjuvant comprising a vaccinia virus vector encoding at least one polypeptide that neutralizes an immune suppressive factor.

2. A method for enhancing an immune response to a vaccine comprising administering a vaccine in combination with the vaccine adjuvant of claim 1 to a subject so that the cells of the subject express the at least one polypeptide thereby enhancing an immune response to the vaccine in the subject.

3. The method of claim 3, further comprising administering a second vaccinia virus vector encoding an immune active cytokine.

4. A kit comprising a formulated vaccine adjuvant of claim 1.
